(19) United States
(12) Patent Application Publication
(10) Pub. No.: US 2010/0130883 A1
(43) Pub. Date: May 27, 2010

(54) In-Vivo Non-Invasive Bioelectric Impedance Analysis of Glucose-Mediated Changes in Tissue

(76) Inventors: Scott E. Carpenter, Pendleton, IN (US); Jilane G. Faigh, Elkhart, IN (US); Kathleen A. Black, Bristol, IN (US)

Correspondence Address: NIXON PEABODY LLP, 300 S. Riverside Plaza, 16th Floor, CHICAGO, IL, 60606-6613, US

(21) Appl. No.: 12/086,234

(22) PCT Filed: Dec. 14, 2006
(86) PCT No.: PCT/US2006/047901

§ 371 Date: Dec. 1, 2009

Related U.S. Application Data
(60) Provisional application No. 60/751,241, filed on Dec. 16, 2005.

(51) Int. Cl. A61B 5/053

(52) U.S. Cl. 600/547

(57) ABSTRACT

A non-invasive, in vivo method for measuring glucose-mediated changes in tissue is disclosed. The method comprises the act of providing a system for directly or indirectly measuring impedance values. The method further comprises the act of providing at least three electrodes and corresponding electrode pads. The electrodes are connected to the system. The method further comprises the act of contacting the electrode pads to a user's skin. The method further comprises the act of contacting each of the at least four electrodes to a corresponding electrode pad. The method further comprises the act of applying an alternating current. The method further comprises the act of determining the correlation between the glucose concentration in the tissue and the measured changes in impedance.

### FIELD OF THE INVENTION

The present invention relates generally to an in-vivo method for measuring glucose-mediated changes in tissue using in-vivo bioelectric impedance analysis.

### BACKGROUND OF THE INVENTION

The quantitative determination of analytes in body fluids is of great importance in the diagnoses and maintenance of certain physiological abnormalities. In particular, determining glucose in body fluids is important to diabetic individuals who must frequently check the glucose level in their body fluids to regulate the glucose intake in their diets.

In one current type of blood glucose testing system, test sensors are used to test a sample of blood. The testing end of the test sensor is placed into the blood that has, for example, accumulated on a person's finger after the finger has been pricked. Blood samples are often taken from a fingertip of a test subject because of the high concentration of capillaries, which can provide an effective blood supply. The blood is drawn into a capillary channel that extends in the test sensor from the testing end to the reagent material by capillary action so that a sufficient amount of blood is drawn into the test sensor. A voltage is applied, causing the glucose in the blood to then chemically react with the reagent material in the test sensor, resulting in an electrical signal indicative of the glucose level in the blood. This signal is supplied to a sensor-dispensing instrument, or meter, via contact areas located near the rear or contact end of the test sensor and becomes the measured output.

Drawing blood each time a glucose reading is desired is an inconvenient and invasive procedure. Moreover, taking a blood sample is undesirable because of the resulting pain and discomfort often experienced by test subjects.

A number of methods have been proposed for non-invasive measurement of blood glucose. One of these methods is spectroscopy (e.g., infrared or Raman spectroscopy), which is advantageous because of its specificity for glucose. In spectroscopic techniques, the ability to develop accurate models for glucose prediction, as well as the ability to demonstrate calibration transfer between subjects, is governed by dynamic changes in the tissue being measured. For example, glucose is generally located in a complex matrix of tissue. Using spectroscopy, peaks or components of a signal corresponding to a target glucose molecule may be identified. The peaks or components generally vary in intensity according to the concentration of glucose

One obstacle pertaining to spectroscopic methods is the possible variation of tissue composition such as, for example, skin temperature changes, skin hydration, and/or hemoglobin concentration. These variations may impact photon migration and scattering mechanisms on which the spectroscopic methods are based, thus impairing the accuracy of predictive models generated by these methods. Thus, frequent calibration may be required and irreproducible data may be generated.

It would be desirable to have a method to correct spectroscopic measurements by compensating for the dynamic changes that occur in the glucose-containing tissue matrix.

### SUMMARY OF THE INVENTION

According to one embodiment of the present invention, a non-invasive, in vivo method for measuring glucose-mediated changes in tissue is disclosed. The method comprises the act of providing a system for directly or indirectly measuring impedance values. The method further comprises the act of providing at least three electrodes and corresponding electrode pads. The electrodes are connected to the system. The method further comprises the act of contacting the electrode pads to a user's skin. The method further comprises the act of contacting each of the at least four electrodes to a corresponding electrode pad. The method further comprises the act of applying an alternating current. The method further comprises the act of determining the correlation between the glucose concentration in the fluid and the measured changes in impedance.

According to another embodiment of the present invention, a method for improving or enhancing a spectroscopic technique for monitoring glucose is disclosed. The method comprises the act of performing a spectroscopic technique. The method further comprises the act of applying bioelectric impedance analysis to determine a correction factor based on monitored changes in tissue composition. The method further comprises the act of applying the correction factor to the spectroscopic technique.

The above summary of the present invention is not intended to represent each embodiment, or every aspect, of the present invention. Additional features and benefits of the present invention are apparent from the detailed description and figures set forth below.

### BRIEF DESCRIPTION OF THE DRAWINGS

FIG. 1 is a diagram of a system of the present invention according to one embodiment.

FIG. 2 is a line graph showing the relationship between glucose levels and impedance values over time.

FIG. 3 is line graph plotting the change in impedance versus the change in glucose.

FIG. 4 is a line graph showing resistance slopes at various fluid drip rates.

### DESCRIPTION OF ILLUSTRATED EMBODIMENTS

The present invention is directed to a method of measuring glucose-mediated changes in tissue. The method uses a low frequency bioelectric impedance analysis (“BIA”) meter for in-vivo monitoring of tissue parameters affected by the presence or absence of glucose. The tissue may include fluid such as blood, interstitial fluid (ISF), and/or extracellular fluid. This method does not directly measure glucose. However, the detected changes in impedance correlate closely with increases and decreases in glucose concentration, as well as the rate of change of glucose concentration.

The invention uses an indirect, non-invasive method of analysis. The invention may utilize a commercially available BIA meter to rapidly monitor changes in small volumes of tissue composition due to the presence or absence of glucose

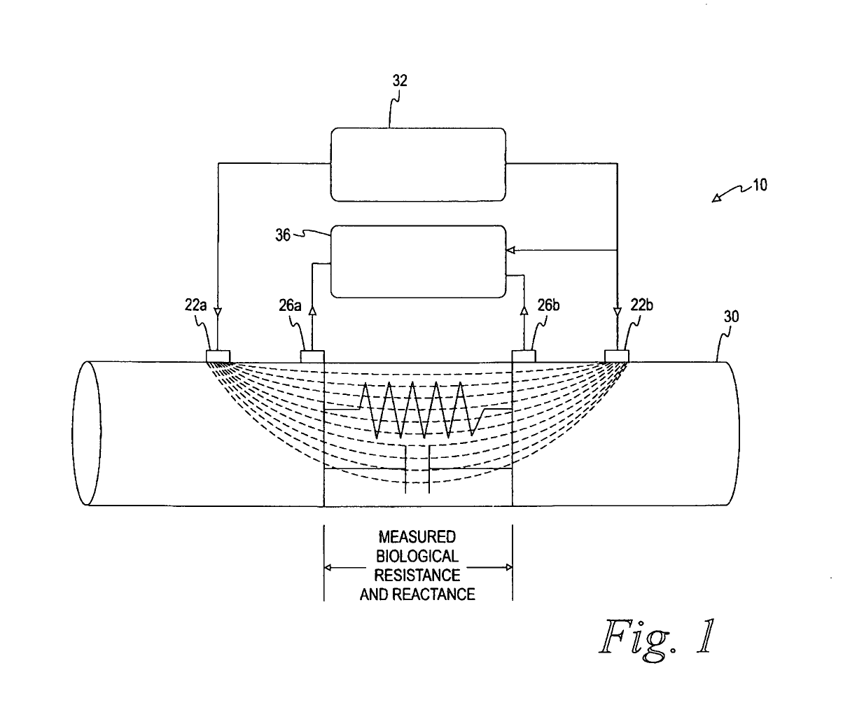

Referring to FIG. 1, a system 10 for taking localized BIA measurements is shown according to one embodiment. The system 10 utilizes a tetrapolar surface electrode configuration including two current source electrodes 22a,b and two detecting electrodes 26a,b. The system 10 further includes four surface electrode pads. The electrode pads include a conductive metal disc at the center of the pads coated with a conductive gel and surrounded by adhesive at the outer part of the pads. The electrode pads are applied to a surface 30. The surface 30 is generally an area of skin, which may be located on various portions of the body including, but not limited to, a forearm, abdomen, an ear lobe, a finger, or a web area generally located between an index finger and thumb. The skin area may be prepared prior to making contact with the electrode pads. For example, the skin may be washed with soap and water, an abrasive, and/or alcohol to remove dirt, dead skin cells, and/or impurities that may alter the impedance measurements. The skin may also be shaved so that hair on the skin does not interfere with the measurements. A first end of each surface electrode 22a,b, 26a,b is coupled to each electrode pad. A second end of each current source electrode 22a,b is coupled to a constant current source 32. A second end of each detecting electrode 26a,b is coupled to an alternating current (“AC”) volt meter 36. The constant current source 32, the AC volt meter 36, and the surface electrodes may be housed within a meter, such as a BIA meter, a wristwatch, or other suitable housing. In such an embodiments, the electrodes may extend through apertures formed in the housing. It is desirable to use at least four surface electrodes to reduce or eliminate problems with impedance at the electrode-skin interface. By using at least four surface electrodes, the impedance of the skin and the electrode polarization impedance do not effect the measurements since negligible current is drawn through the skin by the passively coupled input circles. It is contemplated that additional surface electrodes may be used. In such embodiments, a system typically has a plurality of source electrodes and detecting electrodes.

The constant current source 32 applies a current that runs through the plurality of current source electrodes 22a,b to the surface 30. Typically, currents less than 1 mA are employed (e.g., 800 μA). The current is typically applied at one or more frequencies ranging from about 1 kHz to about 1 MHz. The embodiment presented employs a single frequency of 50 kHz. In this relatively low frequency range, current flows primarily through extracellular fluids, and cell membranes cannot be easily polarized. The current and frequency may also be selected to satisfy safety standards such as AAMI and UL-544 safety standards for a medical device.

After the current has been applied, the plurality of detecting electrodes 26a,b monitors the reactance and resistance of the localized tissue. The impedance of the tissue is determined from the reactance and resistance of the tissue. Changes in glucose concentrations in fluid generally correlate with changes in the measured impedance of the tissue. Thus, a user may determine whether his glucose level has changed significantly using the system and method of the present invention.

One example of a commercial BIA meter that may be used to demonstrate this invention is the Physiological Event Analyzer (PEA) from RJL Systems (Clinton Township, Mich.). The BIA meter directly measures serial resistance and serial reactance over a range of 0-1,000 ohms with a resolution of 0.1 ohms. The instrument may also calculate and report impedance, phase angle, parallel resistance, parallel reactance, and capacitance. The BIA meter and test leads are configured for tetrapolar electrode measurements. It is contemplated that other meters may also be used.

### EXAMPLES

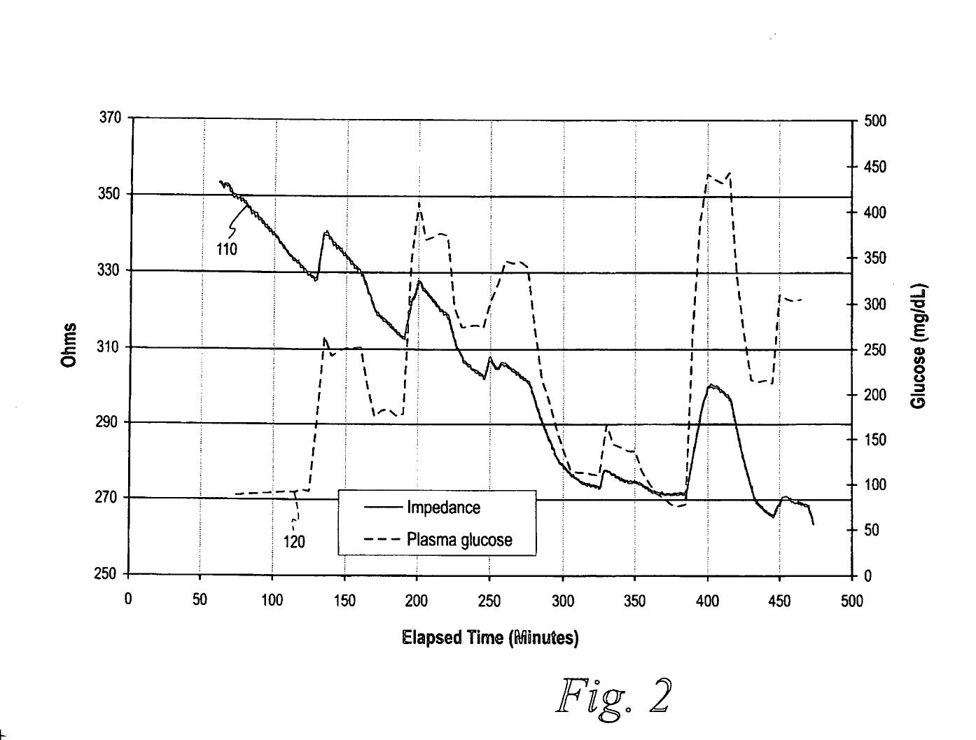

Referring to FIGS. 2-4, a study was performed comparing changes in glucose levels to changes in impedance values using the system and method described above. Data were collected using the present invention to monitor changes in glucose in localized tissue. The data shown in FIGS. 2-4 were derived from a laboratory glucose-clamping study using an animal. The study showed a distinct relationship between measured impedance and infused glucose levels.

The animal used in the study of FIGS. 2-4 was given anesthesia, and its vital signs (e.g., heart rate) were monitored throughout the testing process according to board-reviewed experimental and safety protocols. Surface electrode pads were placed on the animal's skin on the topside of its ear. A small area of hair was shaved to facilitate placement of the electrodes. As described in the system of FIG. 1 above, a tetrapolar electrode configuration, including two current source electrodes and two detecting electrodes, was used to monitor a localized volume of tissue. A first end of each of the two current source electrodes and a first end of each of the two detecting electrodes were placed on respective electrode pads. The detecting electrodes were positioned between the current source electrodes. A second end of each of the current source electrodes was coupled to a constant current source. A second end of each of the detecting electrodes was coupled to an AC volt meter. A controllable syringe pump was used to inject small doses of glucose intravenously into the animal. The amount of glucose injected and/or the rate of infusion was varied, and the effects of the various infusions were monitored.

FIG. 2 showed the changes in impedance correlating with actual blood glucose values measured by a Beckman glucose analyzer, manufactured by Beckman Coulter, Inc. (Fullerton, Calif.). The impedance (ohms) and the glucose values (mg/dL) were plotted against the elapsed time (minutes). The BIA impedance data were plotted and connected by a line 110. The glucose data were plotted and connected by a line 120. The impedance measurements correlate with rapid electrolyte or metabolic changes in tissue due to the presence or absence of glucose. As shown in FIG. 2, changes in impedance correspond to changes in the amount of glucose injected. For example, when the amount of glucose was significantly increased at about 200 minutes and at about 400 minutes, the impedance value also increased proportionally. The broad slope of the impedance background is directly related to the intravenous drip rate of the isotonic fluid being administered to maintain consistent hydration during the course of the glucose clamping experiment.

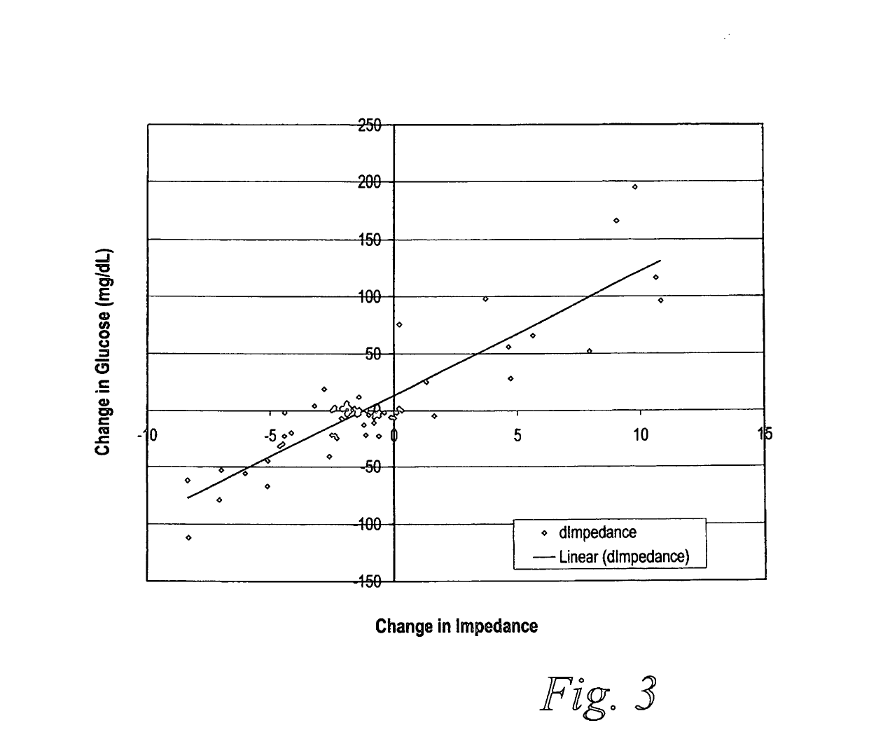

Referring now to FIG. 3, which is based upon the data of FIG. 2, the change in impedance was plotted against the change in blood glucose. Taking the derivative of the impedance signal removes the sloping background to facilitate easier comparison with the changes in glucose. As shown in FIG. 3, there was an observable and statistically significant linear relationship between the change in impedance and the change in blood glucose level.

Further experiments were conducted to verify that impedance changes were associated with changes in glucose rather than administration of other fluids during infusion. For example, saline was injected at various infusion rates at various times to determine whether impedance would be affected. The substitution of saline for glucose during infusion did not produce a change in impedance.

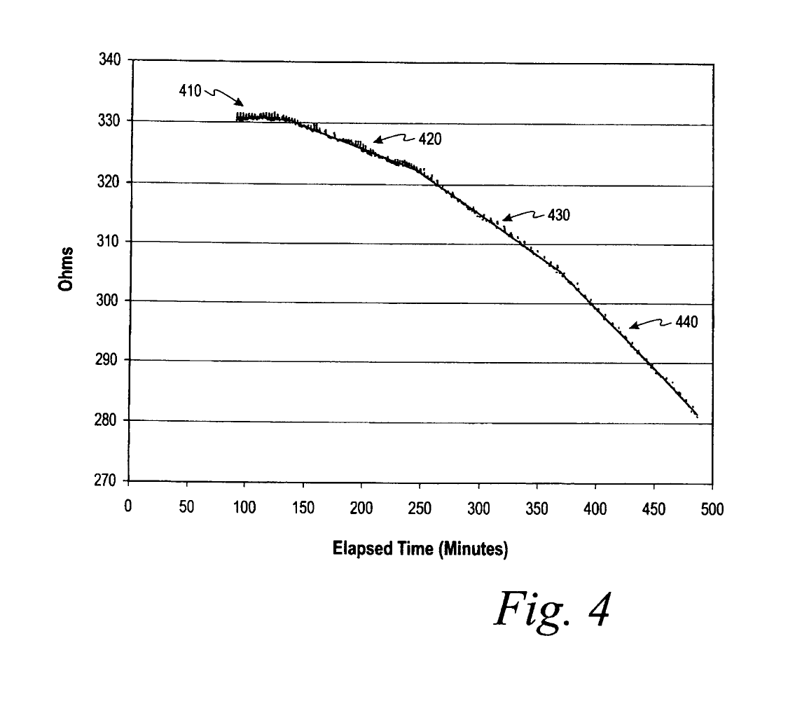

To show that the sloping background is related to the intravenous drip rate of the isotonic replacement fluid, an experiment was conducted in which the drip rate was varied, but no glucose was delivered. Referring now to FIG. 4, the resistance at various replacement fluid drip rates was shown. Resistance (ohms) was plotted against time (minutes), and a best-fit line segment corresponding to the data for each drip rate was determined. First line segment 410 corresponds to the resistance when no fluid was being injected. Second line segment 420 corresponds to the resistance at a fluid drip rate of 60 mL/hr. Third line segment 430 corresponds to the resistance at a fluid drip rate of 80 mL/hr. Fourth line segment 440 corresponds to a fluid drip rate of 100 mL/hr. As shown in FIG. 4, the magnitude of the overall resistance slope increased as the intravenous drip rate increased. For example, the resistance slope at the fluid drip rate of zero was 0.0031. The resistance slope at the fluid drip rate of 60 mL/hr was −0.0729. The resistance slope at the fluid drip rate of 80 mL/hr was −0.1373. Finally, the resistance slope at the fluid drip rate of 100 mL/hr was −0.2033.

According to the present invention, BIA may be used to correct or enhance spectroscopic data that are specific for glucose. Currently, variations in tissue composition often impair the accuracy of predictive spectroscopic models and the ability to demonstrate calibration transfer between subjects. Variations in tissue composition include skin temperature changes, skin hydration, and hemoglobin concentration. These variations may greatly impact photon migration and scattering mechanisms, thus impairing the accuracy of predictive models. Therefore, frequent calibration may be required, and irreproducible data may be generated. Because BIA directly monitors the properties of the tissue matrix containing glucose, BIA measurements may be used to correct or enhance spectroscopic calibration models. This method is a multi-sensor approach that leverages the strengths of multiple methods to achieve results that cannot be obtained by either method alone. In practice, the BIA and spectroscopic sensors simultaneously monitor the same volume of tissue. By monitoring the changes in the tissue using BIA, a correction may be applied to account for the changes in the tissue, thus increasing the accuracy of the spectroscopic models.

### Alternate Embodiment A

A non-invasive, in vivo method for measuring glucose-mediated changes in tissue, the method comprising the acts of:

providing a system for directly or indirectly measuring impedance values;

providing at least three electrodes and corresponding electrode pads, the electrodes being connected to the system;

contacting the electrode pads to a user's skin;

contacting each of the at least three electrodes to a corresponding electrode pad;

applying an alternating current; and

determining the correlation between the glucose concentration in the fluid and the measured changes in impedance.

### Alternative Process B

The method of Alternative Process A, wherein the system for measuring impedance calculates the impedance based on measured resistance and reactance values.

### Alternative Process C

The method of Alternative Process A, wherein the alternating current is applied at a single frequency.

### Alternative Process D

The method of Alternative Process A, wherein the alternating current is applied at multiple frequencies applied in a serial fashion.

### Alternative Process E

The method of Alternative Process A, wherein the at least three electrodes is four electrodes, the four electrodes including two current source electrodes and two detecting electrodes.

### Alternative Process F

The method of Alternative Process A, wherein the applied current is less than 1 mA.

### Alternative Process G

The method of Alternative Process A, wherein the frequency of the alternating current being applied is from about 1 kHz to about 1 MHz.

### Alternative Process H

The method of Alternative Process A, wherein the meter includes a volt meter and an alternating-current source.

### Alternative Process I

The method of Alternative Process A, wherein the fluid is blood, interstitial fluid, and extracellular fluid.

### Alternative Process J

The method of Alternative Process A, wherein the frequency ranges from about 25 kHz to about 75 kHz.

### Alternative Process K

A non-invasive, in vivo method for measuring glucose-mediated changes in tissue, the method comprising the acts of:

providing a system for directly or indirectly measuring impedance values;

providing two current source electrodes, the electrodes being coupled to the system;

providing two detecting electrodes, the electrodes being coupled to the system;

coupling the current source electrodes and the detecting electrodes to a user's skin;

applying an alternating current;

measuring at least one resistance value and at least one reactance value;

calculating the impedance based on the measured resistance and reactance values; and

determining the correlation between the glucose concentration in the tissue and the measured changes in impedance.

### Alternative Process L

The method of Alternative Process K, wherein the two current source electrodes and the two detecting electrodes are coupled to respective electrode pads, the electrode pads being coupled to the user's skin.

### Alternative Process M

The method of Alternative Process K, wherein the alternating current is applied at a single frequency.

### Alternative Process N

The method of Alternative Process K, wherein the alternating current is applied at multiple frequencies applied in a serial fashion.

### Alternative Process O

The method of Alternative Process K, wherein the applied current is less than 1 mA.

### Alternative Process P

The method of Alternative Process K, wherein the frequency of the alternating current being applied is from about 1 kHz to about 1 MHz.

### Alternative Process Q

The method of Alternative Process K, wherein the meter includes a volt meter and an alternating-current source.

### Alternative Process R

The method of Alternative Process K, wherein the tissue includes blood, interstitial fluid, extracellular fluid, or combinations thereof.

### Alternative Process S

The method of Alternative Process K, wherein the frequency ranges from about 25 kHz to about 75 kHz.

### Alternative Process T

A method for improving or enhancing a spectroscopic technique for monitoring glucose, the method comprising the acts of:

performing a spectroscopic technique;

applying bioelectric impedance analysis simultaneously to determine a correction factor based on monitored changes in tissue composition; and applying the correction factor to the spectroscopic technique.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents, and alternatives falling within the spirit and scope of the invention as defined by the appended claims.

1. A transdermal test sensor assembly adapted to determine an analyte concentration of a fluid sample, the test sensor assembly comprising:
a sensor support including at least one reservoir adapted to hold a liquid;
a test sensor being coupled to the sensor support, the test sensor forming at least one aperture therein, at least a portion of the at least one aperture being adjacent to the at least one reservoir; and
a hydrogel composition positioned on the test sensor, the hydrogel composition being linked to the at least one reservoir via the at least one aperture.

2. The assembly of claim 1, wherein the at least one reservoir further includes a liquid.

3. The assembly of claim 2, wherein the hydrogel includes a solvent, the liquid of the at least one reservoir includes a solvent, the solvent percentage of the liquid being greater than the solvent percentage of the hydrogel.

4. The assembly of claim 1, wherein the sensor support further includes a recessed area having dimensions generally similar to dimensions of the test sensor, the recessed area being adjacent to the test sensor, the at least one reservoir being positioned within the recessed area.

5. The assembly of claim 1, wherein the assembly further comprises a coupling mechanism for coupling the test sensor assembly to an analyte-testing instrument.

6. The assembly of claim 1, wherein the hydrogel composition comprises at least one monomer and a solvent.

7. A transdermal analyte-testing assembly adapted to determine an analyte concentration of a sample, the analyte-testing assembly comprising:
a sensor support including at least one reservoir adapted to hold a liquid;
a test sensor being coupled to the sensor support, the test sensor forming at least one aperture therein, at least a portion of the at least one aperture being adjacent to the at least one reservoir;
a hydrogel composition being linked to the at least one reservoir via the at least one aperture; and
an analyte-testing instrument coupled to the sensor support, the analyte-testing instrument being adapted to determine an analyte concentration of a sample.

8. The assembly of claim 7, wherein the at least one reservoir further includes a liquid.

9. The assembly of claim 7, wherein the hydrogel includes a solvent, the liquid of the at least one reservoir includes a solvent, the solvent percentage of the liquid being greater than the solvent percentage of the hydrogel.

10. The assembly of claim 7, wherein the sensor support further includes a recessed area having dimensions generally similar to dimensions of the test sensor, the recessed area being adjacent to the test sensor, the at least one reservoir being positioned within the recessed area.

11. The assembly of claim 7, wherein the hydrogel composition comprises at least one monomer and a solvent.

12. The assembly of claim 7, wherein the analyte-testing instrument is adapted to determine the analyte concentration at pre-selected time intervals.

13. A non-invasive method of determining a concentration of at least one analyte in a body fluid, the method comprising the acts of:
providing a transdermal test sensor assembly including a sensor support, a test sensor, and a hydrogel composition, the test sensor support including at least one reservoir, the at least one reservoir including a liquid, the test sensor being coupled to the sensor support, the test sensor forming at least one aperture therein, at least a portion of the at least one aperture being adjacent to the at least one reservoir, the hydrogel composition being linked to the at least one reservoir via the at least one aperture;
contacting the transdermal sensor to an area of skin such that the hydrogel composition is positioned between the skin and the test sensor;
coupling an analyte-testing instrument to the transdermal test sensor assembly; and
determining the concentration of the analyte using the analyte-testing instrument.

14. The method of claim 13, wherein the area of skin is pre-treated.

15. The method of claim 13, wherein the act of determining the concentration of the analyte using the analyte-testing instrument is repeated at pre-selected time intervals.

16. The method of claim 13, wherein the hydrogel includes a solvent, the liquid of the at least one reservoir includes a solvent, the solvent percentage of the liquid being greater than the solvent percentage of the hydrogel.

17. The method of claim 13, wherein the sensor support further includes a recessed area having dimensions generally similar to dimensions of the test sensor, the recessed area being adjacent to the test sensor, the at least one reservoir being positioned within the recessed area.

18. The method of claim 13, wherein the hydrogel composition comprises at least one monomer and a solvent.
